(19) United States
(12) Patent Application Publication
(10) Pub. No.: US 2022/0143053 A1
(43) Pub. Date: May 12, 2022

(54) ANTI-MICROBIAL METHODS AND MATERIALS

(71) Applicant: The General Hospital Corporation, Boston, MA (US)

(72) Inventors: Orhun K. Muratoglu, Cambridge, MA (US); Ebru Oral, Newton, MA (US); Dmitry Gil, Quincy, MA (US)

(21) Appl. No.: 17/437,912

(22) PCT Filed: Mar. 24, 2020
(86) PCT No.: PCT/US2020/024497

§ 371 Date: Sep. 10, 2021

Related U.S. Application Data
(60) Provisional application No. 62/824,206, filed on Mar. 26, 2019.

(51) Int. Cl. A61K 31/702; A61K 47/32; A61K 31/445; A61K 31/407; A61K 31/196; A61K 31/245; A61K 31/065; A61K 31/121; A61K 31/167; A61K 38/14

(52) U.S. Cl. CPC A61K 31/702; A61K 47/32; A61K 31/445; A61K 31/407; A61K 31/196; B29K 2023/0683; A61K 31/065; A61K 31/121; A61K 31/167; A61K 38/14; A61K 31/245

(57) ABSTRACT

The present invention provides methods for making and using antibacterial polymeric materials loaded with additives, as well as antibacterial materials comprising additives. Certain additives or combinations of additives show unexpected combinatorial or synergistic antibacterial activity. The invention also provides medical devices comprised of antibacterial polymeric materials, and methods of making and using such devices, which can have unexpected combinatorial or synergistic antibacterial activity.

### CROSS REFERENCE TO RELATED APPLICATIONS

This application claims priority to U.S. Provisional Application No. 62/824,206, filed Mar. 26, 2019, the contents of which are hereby incorporated by reference into this application.

### STATEMENT REGRADING FEDERALLY FUNDED RESEARCH

This work was supported by the Office of the Assistant Secretary of Defense for Health Affairs, through the Peer Reviewed Medical Research Program under Award No. W81XWH-17-1-0614.

### FIELD OF THE INVENTION

The present invention relates to methods for making antibacterial polymeric materials loaded with additives Some additives or combinations of additives show unexpected combinatorial or synergistic antibacterial activity. The invention also relates to making medical devices comprised of antibacterial polymeric materials.

### BACKGROUND OF THE INVENTION

The potential for post-operative infection exists for all areas of medicine involving the implantation of foreign objects. Annually more than 30 million bladder catheters are used in the US and the infection rate is estimated at 10-30%. More than 300,000 cardiac pacemakers are used and an infection rate of up to 7% is observed. Roughly 3% of the 85,000 mechanical heart valves are infected after implantation. More than 3 million fracture fixation devices and 1 million total joint pairs are implanted every year, and the infection rate can reach up to 10-30% and 3%, respectively (Darouiche, R O. Device-Associated Infections: A Macroproblem that Starts with Microadherence. Clinical Infectious Diseases, 2001, 33:1567-1572). These rates generally refer to primary surgeries, i.e. first-time implantations. The rate of infection associated with re-implanted devices is several times that for first-time implants.

Orthopaedic implant-associated infections are of particular importance as they result in high rates of morbidity and mortality. Periprosthetic joint infection (PJI) is a major factor hindering the longevity of joint replacements. Although its prevalence rate is low (1-2%), PJI accounts for 20% of revisions and the associated mortality rate can reach 25%, which is comparable to that of some types of cancer (Kurtz S M, Lau E, Schmier J, Ong K L, Zhao K, Parvizi J. Infection Burden for Hip and Knee Arthroplasty in the United States. J Arthroplasty. 2008; 23(7):984-91)

There are multiple reasons why surgical infections are persistent and associated with high morbidity and mortality. During implantation, the surgical site is exposed to numerous opportunistic bacteria introduced through the incision. In their seminal work on biofilm formation, Gristina et al. suggested that the genesis of an infection is highly dependent on the outcomes of the “race to the surface” between host immune cells and pathogens. If the “race” is lost and bacteria reaches an artificial surface before the immune cells, an implant gets colonized, and a biofilm is formed (A. Gristina, Biomaterial-centered infection: microbial adhesion versus tissue integration, Science, 237 (1987) 1588-1595). It is estimated that biofilms are involved in approximately 80% of surgical infections.

Current clinical approaches to address surgical infections rely heavily on post-surgical antibiotic prophylaxis involving systemic administration of various antibiotics, such as gentamicin, tobramycin, vancomycin, etc. If infection is diagnosed, the treatment options often involve removal of the implant, irrigation and debridement of the infected area, re-implantation of a new implant, and prolonged systemic administration of antibiotics (Kurtz S M, Lau E, Schmier J, Ong K L, Zhao K, Parvizi J. Infection Burden for Hip and Knee Arthroplasty in the United States. J Arthroplasty. 2008; 23(7):984-91). The efficacy of these treatment strategies depends heavily on the bioavailability of systemic antibiotics at the tissue-implant interface. Therefore, systemically administered antibiotics show high efficacy in irradiating renal and cardio-vascular infections. Yet, tissues with low blood flow, such as bone and cartilage, were shown to be a haven for infections even after long-term antibiotic regiments, as the bioavailability of systemic antibiotics in the bone/implant interface is very low and inefficient. For example, vancomycin concentration in the bone is only ˜20% of that in the serum (Rybak M J, Lomaestro B M, Rotschafer J C, Moellering R C, Craig W A, Billeter M, et al. Vancomycin Therapeutic Guidelines: A Summary of Consensus Recommendations from the Infectious Diseases Society of America, the American Society of Health-System Pharmacists, and the Society of Infectious Diseases Pharmacists. Clin Infect Dis 2009; 49(3):325-327). High local concentrations of antibiotics have the potential to improve the outcomes of revision surgeries. For periprosthetic joint infections (PJI) the gold-standard approach is a two-stage revision comprised of the removal of all components and the placement of an antibiotic-impregnated bone cement (AIBC) in the joint space in a first surgery—an implant that provides local sustained delivery of antibiotics for up to 6 months. Yet, the success rate of the gold-standard two-stage surgery is <80% (Cochran A R, Ong K L, Lau E, Mont M A, Malkani A L. Risk of Reinfection After Treatment of Infected Total Knee Arthroplasty. J Arthroplasty, 2016; 31(9):156-161). The incidence of the secondary PJI, as well as the resistance of the encountered bacteria to commonly used antibiotics is increasing at a steady rate. Thus, there is a need to further improve the efficacy of post-surgical antibacterial regimens.

Given that all surgical interventions involve a degree of discomfort, and in the vast majority of cases, painful, the use of analgesics is vital during and after the surgery. In current clinical practice several classes of analgesics, such as sodium-channel blockers, non-steroidal anti-inflammatory drugs (NSAIDs), and opioids are often administrated concurrently to relieve pain. Depending on the type of surgery, pain relieve therapy can last from several hours to several weeks. For example, Exparel™ (liposome encapsulated bupivacaine) is often administrated after several types of surgeries, such as total joint arthroplasty, hernioplasty, mammoplasty, etc. This formulation provides extended local release of bupivacaine (sodium-channel blocker) for at least 72 hours (S. Cohen, Extended pain relief trial utilizing infiltration of Exparel, a long-acting multivesicular liposome formulation of bupivacaine: a Phase IV health economic trial in adult patients undergoing open colectomy, J. Pain Res. 6 (2012) 567-572).

Several studies showed that certain analgesics possess measurable antimicrobial activity against multiple bacterial pathogens, including Escherichia coli, Pseudomonas aeruginosa, and various strains of Staphylococci—pathogens causing most of the implant-associated infections (O. N. Aydin, M. Eyigor, N. Aydin, Antimicrobial activity of ropivacaine and other local anaesthetics, Eur. J. Anaesthesiol. 18 (2001) 687-694). For example, bupivacaine and lidocaine (sodium channel blockers) were shown to possess measurable antibacterial effects against methicillin-sensitive S. aureus—bacteria associated with most PJI (Gil D, Grindy S, Muratoglu O, Bedair H, Oral E. Antimicrobial Effect of Anesthetic-Eluting Ultra-High Molecular Weight Polyethylene for Post-Arthroplasty Antibacterial Prophylaxis. J Orthop Res 2019; 1-10). Ibuprofen, aspirin, diclofenac and mefenamic acid (NSAIDs) also yielded measurable antibacterial activity against Bacillus cereus, MSSA, MRSA, Escherichia coli, Klebsiella pneumoniae, Pseudomonas aeruginosa, Enterobacter aerogenes, and Salmonella Choleraesuis. A most promising aspect of using analgesics as part of antibacterial prophylaxis is the possibility of increasing the efficiency of antibacterial treatments without increasing safety risks to the patients, as these drugs are already in wide peri-surgical use for anesthesia and analgesia.

In clinical practice, various types of analgesics are used concurrently with conventional antibiotics especially in the immediate peri-surgical period where the infection risk is the highest. The effects of the concurrent use of these drugs on their antibacterial profiles and efficacy are unknown.

Typically, analgesics and other therapeutics are administered via bolus injection. For example, lidocaine, bupivacaine, and ketorolac are administered peri-particularly via bolus injections (120 mg, 150 mg, and 30 mg, respectively) to control post-arthroplasty pain (Karlsen A P H, Wetterslev M, Hansen S E, Hansen M S, Mathiesen O, Dahl J B. 2017. Postoperative pain treatment after total knee arthroplasty: A systematic reviewPLoS ONE). The systemic administration of these drugs is not expected to yield antibacterial effects due to insufficient concentrations at the surgical site, but the doses can be designed to be sufficiently high in a local delivery device to provide antibacterial effects against implant-associated infections. In addition, it is not possible to sustain desired ratios of drug concentrations when therapeutic agents are administered simultaneously. The long-term effects of the simultaneous local administration of multiple agents for a desired effect can only be achieved through incorporating them in a delivery device. Combinations of therapeutics can be delivered using drug-eluting polymeric systems, such as biodegradable hydrogels and/or ultra-high molecular weight polyethylene.

The current invention describes methods of making antibacterial polymeric materials and antibacterial medical devices made from such polymeric materials. The antibacterial polymeric materials are made by the approach of incorporating multiple therapeutic agents at concentrations where they will exhibit unexpected combinatorial antibacterial effects. This approach also enables the alteration and tuning of the antibacterial strength of the polymeric material or medical device.

### Definitions

### Therapeutic Agents/Drugs

As used herein, the term “drug” of “therapeutic agent” refers to a molecule that yields a therapeutic effect when administrated to a living organism. “Analgesics” is a group of drugs that yield pain relief by affecting peripheral and/or central nervous systems. “Anesthetics” is a group of drugs that yield anesthesia—temporary loss of sensation or awareness. Analgesics and anesthetics are often used interchangeably and can be referred to the same drugs. Non-limiting examples of anesthetics or analgesics are methohexital, propofol, thiopental, ketamine, etomidate, isoflurane, fospropofol, sevoflurane, desflurane, diazepam, lorazepam, midazolam, amorbital, thiamylal.

As used herein, the term “sodium channel blocker” refers a class of drugs that act by inhibition of the sodium influx through cell membranes, providing anesthesia. Common sodium channel blockers described herein include, but are not limited to, bupivacaine, lidocaine, procaine, tetracaine, ranolazine, phenytoin, disopyramide, mexiletine, triamterene, lamotrigine, amiloride, moricizine, oxcarbazepine, quinidine, procainamide, tocainide, amiodarone, propafenone, flecainide, encainide, ajmaline, aprindine, tetrodotoxin, eslicarbazepine acetate, pilsicainide, eslicarbazepine, carbamazepine, ethotoin, fosphenytoin, rufinamide, lacosamide, propafenone.

As used herein, the term “nonsteroidal anti-inflammatory drugs” or “NSAIDs” refers to a class of drugs that reduce pain by downregulating inflammatory response. Common nonsteroidal anti-inflammatory drugs described herein include, but not limited to, ketorolac, aspirin, ibuprofen, tolfenamic acid, diclofenac, naproxen, ketoprofen, tolmetin, etodolac, fenoprofen, flurbiprofen, diclofenac, diclofenac/misoprostol, proxicam, indomethacin, sulindac, meloxicam, esomeprazole/naproxen, famotidine/ibuprofen, oxaprozin, mefenamic acid, diflunisal, nabumetone, indomethacin, celecoxib, salsalate, choline, meclofenamate, rofecoxib.

As used herein, the term “antibiotic” refers to a class of drugs that kills or inhibit growth of microorganisms including, but not limited to, bacteria. Common antibiotics described herein include, but not limited to, Amikacin, Gentamicin, Kanamycin, Neomycin, Netilmicin, Tobramycin, Paromomycin, Streptomycin, Spectinomycin, Ansamycins, Geldanamycin, Herbimycin, Rifaximin, Carbacephem, Loracarbef, Carbapenems, Ertapenem, Doripenem, Cefadroxil, Cefazolin, Cephradine, Cephapirin, Cephalothin, Cefalexin, Cefaclor. Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Glycopeptides, Teicoplanin, Vancomycin, Telavancin, Dalbavancin, Lincosamides, Clindamycin, Lincomycin, Lipopeptide, Daptomycin, Macrolides, Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin, Oxazolidinones, Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Sulfonamidochrysoidine, Tetracyclines, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole, Trimethoprim.

The term “antioxidant” refers to what is known in the art as (see, for example, U.S. Pat. No. 8,933,145, WO2001/80778, and U.S. Pat. No. 6,448,315). Alpha- and delta-tocopherol; propyl, octyl, or dodecyl gallates; lactic, citric, ascorbic, tartaric acids, and organic acids, and their salts; orthophosphates, lycopene, tocopherol acetate, curcumin, and resveratrol are non-limiting examples of antioxidants. Antioxidants are also referred as free radical scavengers, include: glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids, including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox family; Irganox and Irganox B families including Irganox 1010, Irganox 1076, Irganox 1330; Irgafos family including Irgafos 168; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, mixtures, derivatives, analogues or conjugated forms of these. Antioxidants/free radical scavengers can be primary antioxidants with reactive OH or NH groups such as hindered phenols or secondary aromatic amines, they can be secondary antioxidants such as organophosphorus compounds or thiosynergists, they can be multifunctional antioxidants, hydroxylamines, or carbon centered radical scavengers such as lactones or acrylated bis-phenols. The antioxidants can be selected individually or used in any combination.

When referred herein, drugs can be present in various forms. Non-limiting examples of the drug forms are powders, aqueous solutions, non-aqueous solutions, gels. Drugs can also be conjugated with counter-ions. Common examples of counterions include, but not limited to, Hydrochloride, Hydrobromide, Acetate, Fumarate, Aspartate, Benzenesulfonate, Benzoate, Besylate, Bicarbonate, Bitartrate, Bromide, Camsylate, Carbonate, Chloride, Citrate, Decanoate, Edetate, Esylate, Fumarate, Gluceptate, Gluconate, Glutamate, Glycolate, Hexanoate, Hydroxynaphthoate, Iodide, Isethionate, Lactate, Lactobionate, Malate, Maleate, Mandelate, Mesylate, Methylsulfate, Mucate, Napsylate, Nitrate, Octanoate, Oleate, Pamoate, Pantothenate, Phosphate, Polygalacturonate, Propionate, Salicylate, Stearate, Acetate, Succinate, Sulfate, Tartrate, Teoclate, Tosylate.

### Bacteria

As used herein, the term “bacteria” refers to a type of biological microorganisms, which possess cell walls, but lacks organelles, and can cause disease. Common bacteria described herein include, but not limited to, methicillin-sensitive S. aureus, methicillin-resistant S. aureus, S. epidermidis. Bacteria can be obtained from the American Type Culture Collection, referred herein as “ATCC”, which is an organization that collects, stores, and distributes reference microorganisms. The term “strain” or “bacterial strain” refers to a subtype of bacteria and used for bacterium identification. Common bacteria strains described herein include, but not limited to, ATCC® 12600, ATCC® 12228, ATCC® 14775, which represent two strains of methicillin-sensitive S. aureus and one strain of S. epidermidis, obtained from ATCC.

As used herein, the term “culture medium” or “growth medium” refers to a nutritionally rich medium that allow bacteria proliferation. There can be different types of culture medium including, but not limited to, culture broth, agar, minimal medium. Common broth described herein include, but not limited to, Lysogeny broth, tryptic soy broth, nutrient broth, brain heart infusion broth—medium that is generally used to grow Staphylococci strains. Commercially available broth can be modified with various media supplements. Non-limiting examples of media supplements are 2-mercaptoethanol, amino acid solutions, bovine serum albumin, cholesterol supplements, Chinese hamster ovary supplements, glutamine, insulin, lipid supplements, serum supplements, sodium pyruvate, transferrin, yeast solution, sodium chloride, magnesium chloride, ammonium sulfate. The term “agar” refers to a gel-like medium that is allows growth of bacteria and used to determine bacteria concentration. Common agar described herein include, but not limited to, trypticase soy agar, Lysogeny agar, Nutrient agar, brain heart infusion agar—medium that is generally used to grow Staphylococci strains.

When growing or proliferating, bacteria can be present in four different phases—growth phases. These phases are often referred to as “lag phase”, “log phase”, “stationary phase”, and “death phase”. During the “lag phase” bacteria are adapting to growth conditions, and bacteria proliferation is often minor. During the “log phase” bacteria are actively proliferating. During the “stationary phase” bacteria growth and death rates are similar, resulting in relatively constant bacterial concentration. During the “death phase” bacterial concentration decreases due to the absence of nutrients. Depending on the phase of the bacteria, results of the antibacterial tests can be different. For example, the MIC of a drug A measured against bacteria in lag phase is often lower than that measured against bacteria in log phase.

As used herein, the term “Log 10 reduction” refers to a 10-fold reduction in numbers of viable bacteria present in the broth.

As used herein, the term “spread plate method” refers to a technique used to transfer bacteria from broth to agar, followed with a subsequent incubation at 37° C. overnight to allow visual observation of bacterial colonies. This method is used to quantify bacterial concentration in growth medium. Depending on the bacterial strain, incubation parameters including, but not limited to, temperature and duration can be different.

### Antibacterial Activity and Testing

As used herein, the term “antibacterial activity” refers to the process of killing or inhibiting the growth of bacteria using one or several therapeutic agents. The term “antibacterial activity” is used to quantify the activity of killing of bacteria in the present of one or several drugs. The terms “kill”, or “killing”, “inhibition” refer to a decrease of bacteria concentration in the broth (<1−log 10) over a defined time period. The term “inhibition of growth” refers to an effect when bacterial concentration in the broth is maintained within a change of 1−log 10. The term “bacterial growth” refers to an effect when bacterial concentration in broth increases (≥1−log 10) over a defined time period.

As used herein, the term “antibacterial test” refers to a procedure that allows to assess antibacterial activity of a drug, or combination of drugs against certain type of bacteria. Non-limiting examples of antibacterial tests are the minimum inhibitory concentration test (measurement of drug MIC), minimum bactericidal concentration test (measurement of drug MBC), fractional inhibitory concentration test (assessment of antibacterial activity of a drug combination).

As used herein, the term “minimum inhibitory concentration” or “MIC” refers to the lowest concentration of a drug that prevents visible growth of bacteria over the period of 24 hours when incubated at 37° C.

As used herein, the term “minimum bactericidal concentration” or “MBC” refers to the lowest concentration of a drug that kills 99.9% of bacteria over the period of 24 hours when incubated at 37° C.

As used herein, the term “antibacterial synergy” refers to the effect when two drugs act together to yield antibacterial activity that is higher than if each antibiotic were used individually. For example, an antibacterial synergistic effect occurs when drug A has an MIC of X and drug B has an MIC of Y, and the inhibitory effect on bacteria proliferation is observed when less than X/2 of A and Y/2 of B is used concurrently.

As used herein, the term “additive effect” refers to the effect when antibacterial activity of two drugs is approximately equal to the combined antibacterial activities of individual drugs. For example, an additive effect occurs when drug A has an MIC of X and drug B has an MIC of Y, and the inhibitory effect on bacteria proliferation is observed when more than X/2 of A and Y/2 of B, but less than 2*X of A and 2*Y of B is used concurrently.

As used herein, the term “antagonistic antibacterial effect” refers to the effect when the antibacterial activity of two drugs is less than the combined antibacterial activity of individual drugs. For example, an additive effect occurs when drug A has an MIC of X and drug B has an MIC of Y, and the inhibitory effect on bacteria proliferation is observed when more than 2*X of A and 2*Y of B is used concurrently.

As used herein, the term “fractional inhibitory concentration index” or “FIC” refers to a parameter used to determine the impact on antibacterial activity of the combination of drugs in comparison with the individual drugs. FIC index (ΣFIC) is calculated as follows:

$$Σ FIC = \frac{{MIC}_{A}}{MIC ( A )} + \frac{{MIC}_{B}}{MIC ( B )}$$

Where MIC_{A} is an MIC of drug A in combination, MIC(A) is an MIC of drug A alone, MIC_{B} is an MIC of drug B in combination, MIC(B) is an MIC of drug B alone. Drug combinations show antibacterial synergy if ΣFIC≤0.5; an additive effect is observed if 0.5<ΣFIC<4; and ΣFIC>4 represent antagonistic antibacterial effect.

The results of the antibacterial tests of a drug or drug combination depend largely on the growth medium used for testing. For example, the MIC or FIC values of a drug or drug combination can be different if different types of broth are used. For example, the MIC test conducted in supplemented broth can yield the MIC higher than when the test is conducted in commercially available broth.

Depending on the bacterial strain, the protocol or the parameters of a given antibacterial test can be altered. The parameters that can be changed include, but not limited to, temperature and duration of incubation. If various protocols are used for the same antibacterial test, the results can also be different. For example, the results of the MIC test conducted at 35° C. can be lower than that conducted at 37° C.

### Polymeric Material and Methods of Making Polymeric Material

As used herein, the term “polymeric material” refers to large molecules or macromolecules composed of many repeating subunits. “Polymeric material” includes polyolefins such as polyethylene or polypropylene. Polyethylene can include low density polyethylene(s), and/or linear low-density polyethylene(s) and/or high-density polyethylene(s) and/or ultrahigh molecular weight polyethylene(s) or mixtures thereof. For example, ultra-high molecular weight polyethylene (UHMWPE) refers to linear non-branched chains of ethylene having molecular weights in excess of about 500,000, preferably above about 1,000,000, and more preferably above about 2,000,000. Often the molecular weights can reach about 8,000,000 or more. By initial average molecular weight is meant the average molecular weight of the UHMWPE starting material, prior to any irradiation. See U.S. Pat. Nos. 5,879,400 and 6,641,617, EP0881919, and WO2001/005337. The term “polyethylene article” or “polymeric article” or “polymer” generally refers to articles comprising any “polymeric material” disclosed herein. “Polymeric materials” or “polymers” can also include structural subunits different from each other. Such polymers can be di- or tri- or multiple unit-copolymers, alternating copolymers, star copolymers, brush polymers, grafted copolymers or interpenetrating polymers. They can be essentially solvent-free during processing and use such as thermoplastics or can include a large amount of solvent such as hydrogels. Polymeric materials also include synthetic polymers, natural polymers, blends and mixtures thereof. Polymeric materials also include degradable and non-degradable polymers.

Polymeric materials” or “polymer” also include hydrogels, such as poly (vinyl alcohol), poly (acrylamide), poly (acrylic acid), poly(ethylene glycol), poly(lactic acid)-based hydrogels blends thereof, interpenetrating networks thereof, or hydrogels described in any of the embodiments, which can absorb water such that water constitutes at least 1 to 10,000% of their original weight, typically 100 wt % of their original weight or more or 99% or less of their weight after equilibration in water.

“Polymeric material” or “polymer” can be in the form of resin, flakes, powder, consolidated stock, implant, and can contain additives such as antioxidant(s) or therapeutic agents. The “polymeric material” or “polymer” also can be a blend of one or more of different resin, flakes or powder containing different concentrations of additive(s) such as antioxidants and/or therapeutic agents and/or a chemical crosslinking agents and/or anticross-linking agents and/or crosslinking enhancers. The blending of resin, flakes or powder can be achieved by the blending techniques known in the art. The “polymeric material” also can be a consolidated stock of these blends.

The term “anticross-linking agent” is used to describe additives which can hinder cross-linking when added to be polymeric material. Some free radical scavengers can act as anticross-linking agents. Some other chemicals such as solvents can also act as anticross-linking agents. “Crosslinking enhancer” is used to describe additives which can enhance or increase crosslinking when added to the polymeric material. Some chemicals with unsaturated groups such as acetylene or some solvents can act as crosslinking enhancers.

The products and processes of this invention also apply to various types of polymeric materials, for example, any polypropylene, any polyamide, any polyether ketone, or any polyolefin, including high-density-polyethylene, low-density-polyethylene, linear-low-density-polyethylene, ultra-high molecular weight polyethylene (UHMWPE), copolymers or mixtures thereof. The products and processes of this invention also apply to various types of hydrogels, for example, poly(vinyl alcohol), poly(ethylene glycol), poly(ethylene oxide), poly(acrylic acid), poly(methacrylic acid), poly(acrylamide), copolymers or mixtures thereof, or copolymers or mixtures of these with any polyolefin. Polymeric materials, as used herein, also applies to polyethylene of various forms, for example, resin, powder, flakes, particles, powder, or a mixture thereof, or a consolidated form derived from any of the above. Polymeric materials, as used herein, also applies to hydrogels of various forms, for example, film, extrudate, flakes, particles, powder, or a mixture thereof, or a consolidated form derived from any of the above.

The term “medical device” refers to an instrument, apparatus, implement, machine, implant or other similar and related article intended for use in the diagnosis, treatment, mitigation, cure, or prevention of disease in humans or other animals. An “implantable device” is a medical device intended to be implanted in contact with the human or other animal for a period of time. “Implant” refers to an “implantable medical device” where a medical device, is placed into contact with human or animal skin or internal tissues for a prolonged period of time, for example at least 2 days or more, or at least 3 months or more or permanently. Implants can be made out of metals, ceramic, polymers or combinations thereof. They can also comprise fluids or living tissues in part or in whole. An “implant” can refer to several components together serving a combined function such as “total joint implant” or it can refer to a single solid form such as an “acetabular cup” as a part. The term ‘medical implant’ refers to a medical device made for the purpose of implantation in a living body, for example and animal or human body. The medical implants include but are not limited to acetabular liners, tibial inserts, glenoid components, patellar components, and other load-bearing, articular components used in total joint surgery.

The term “cross-linking” refers to what is known in the art as processes that result in the covalent bonding of the parts of a material, for example polymer chains in a polymeric material.

The term “peroxide” refers to a group of chemicals with the peroxide functional group. General peroxide categories include inorganic peroxides, organic peroxides, diacyl peroxides, peroxyesters, peoxydicarbonates, dialkyl peroxides, ketone peroxides, peroxyketals, cyclic peroxides, peroxymonocarbonates and hydroperoxides.

The term “crosslinking agent” refers to a compound which can cause cross-linking in polymeric materials. Methods of ‘chemical crosslinking’ or cross-linking using crosslinking agent(s) is described in U.S. Pat. No. 10,220,547, US Publication No. 20160215117, and WO2013/151960A2, which are hereby incorporated by reference in their entireties. In other cases, other stimuli may be used to trigger the reaction such as the application of ultraviolet light, heat, pressure or vacuum, contact with a particular solvent, or irradiation or combinations thereof. In this invention, the cross-linking agents used are often those that are commercially available and may contain impurities. In some embodiments, the cross-linking agents may be 100% pure or less. In some embodiments, the cross-linking agents are 80%, 85%, 90%, 91%, 92%, 93%, 94%, 95%, 96%, 97%, 98%, or 99% pure.

The term “irradiation” refers to what is known in the art as exposing a material to radiation, for example ionizing radiation such as a gamma, electron, X-ray or ultraviolet (UV) radiation. ‘Radiation cross-linking’ refers to a radiation process intended to cross-link a material as a result of irradiation, for example exposing UHMWPE to gamma irradiation to cross-link the material. It also refers to the cross-linking in the material that has resulted from a radiation process. The radiation dose used can be from 0.0001 kGy to 100,000 kGy, or any value therebetween, or 0.1 kGy to 1000 kGy, or from 1 kGy to 1000 kGy, or from 10 kGy to 1000 kGy, or from 25 kGy to 1000 kGy, or from 50 kGy to 1000 kGy, or from 100 kGy to 1000 kGy, or from 1 kGy to 300 kGy, or about 65 kGy, or about 75 kGy, or about 85 kGy, or about 100 kGy, or about 150 kGy, or about 175 kGy, or about 200 kGy. The radiation dose rate can be from 0.001 kGy/min to 100,000 kGy/min, or any value therebetween, or from 0.1 kGy/min to 100 kGy/min, or from 1 kGy/min to 50 kGy/min, or about 25 kGy/min, or about 10 kGy/min, or about 100 kGy/min. Irradiation can be done in air, in vacuum, or partial gas environments, for example mixtures of oxygen and nitrogen. It can also be done in inert gas or partial inert gas. It can also be done at ambient temperature, or below or above ambient temperature. It can be done at elevated temperatures above ambient temperature. Irradiation temperature can be from −100° C. to 1000° C., or any value therebetween, or from 0° C. to 500° C. or from 20° C. to 200° C. or from 25° C. to 150° C., or at about 25° C., or about 70° C., or about 100° C., or about 120° C., or about 125° C. Methods of “exposing to radiation” or “irradiation” are described, for example in U.S. Pat. No. 7,381,752 (Muratoglu), U.S. Pat. No. 7,858,671 (Muratoglu et al.) and U.S. Pat. No. 6,641,617 (Merrill et al.). Also, methods of irradiation and treatments after irradiation are described, for example in U.S. Pat. No. 7,431,874 (Muratoglu et al.), U.S. Pat. No. 6,852,772 (Muratoglu et al.), U.S. Pat. No. 8,420,000 (Muratoglu et al.), U.S. Pat. No. 8,461,225 (Muratoglu et al.) and U.S. Pat. No. 8,530,057 (Muratoglu et al.).

The term “blending” refers to what is known in the art; that is, mixing of different components, often liquid and solid or solid and solid to obtain a homogeneous mixture of said components. Blending generally refers to mixing of a polymeric material in its pre-consolidated form with an additive. If both constituents are solid, blending can be done by using other component(s) such as a liquid to mediate the mixing of the two components, after which the liquid is removed by evaporating. If the additive is liquid, for example α-tocopherol, then the polymeric material can be mixed with large quantities of the said liquid. This high concentration blend can be diluted down to desired concentrations with the addition of lower concentration blends or virgin polymeric material without the additive to obtain the desired concentration blend. This technique also results in improved uniformity of the distribution of the additive in the polymeric material. Methods of blending additives into polymeric material are described, for example in U.S. Pat. Nos. 7,431,874, 9,168,683, 8,425,815, 9,273,189, and WO2007/024684A2 (Muratoglu et al.).

The term “consolidation” refers generally to processes used to convert the polymeric material resin, particles, flakes, i.e. small pieces of polymeric material into a mechanically integral large-scale solid form, which can be further processed, by for example machining in obtaining articles of use such as medical implants. Methods such as injection molding, extrusion, compression molding, iso-static pressing (hot or cold), or other methods known in the art can be used. In the present invention, consolidation of layers of polymeric material having different additives is described.

Consolidation can be performed by “compression molding”. In some instances, consolidation can be interchangeably used with compression molding. The molding process generally involves: (i) heating the polymeric material to be molded; (ii) pressurizing the polymeric material while heated, (iii) keeping at temperature and pressure; (iv) cooling down and releasing pressure.

Heating of the polymeric material can be done at any rate. Temperature can be increased linearly with time or in a stepwise fashion or at any other rate. Alternatively, the polymeric material can be placed in a pre-heated environment. In some embodiments, the polymeric material is placed into a mold for consolidation and he process (steps i-iv) is started without pre-heating. The mold for the consolidation can be heated together or separately from the polymeric material to be molded. Steps (i) and (ii), i.e. heating and pressurizing before consolidation can be done in multiple steps and in any order. For example, polymeric material can be pressurized at room temperature to a set pressure level 1, after which it can be heated and pressurized to another pressure level 2, which still may be different from the pressure or pressure(s) in step (iii). Step (iii), where a high temperature and pressure are maintained is the ‘dwell period’ where a major part of the consolidation takes place. One temperature and pressure or several temperatures and pressures can be used during this time without releasing pressure at any point. For example, dwell temperatures in the range of 135 to 350° C. and dwell pressures in the range of 0.1 MPa to 100 MPa or up to 1000 MPa, or any value therebetween, can be used. The dwell time can be from 1 minute to 24 hours, more preferably from 2 minutes to 1 hour, most preferably about 10 minutes. The temperature(s) at step (iii) are termed ‘dwell’ or ‘molding’ temperature(s). The pressure(s) used in step (iii) are termed ‘dwell’ or ‘molding’ pressure(s). The order of cooling and pressure release (step iv) can be used interchangeably. In some embodiments the cooling and pressure release may follow varying rates independent of each other. In some embodiments, consolidation of polymeric resin or blends of the resin with additive(s) are achieved by compression molding. The dwell temperature and dwell time for consolidation can be changed to control the amount of integration.

Compression molding can also follow “layering” of different polymeric material; in these instances, it is termed “layered molding”. This refers to consolidating a polymeric material by compression molding one or more of its pre-molded and resin forms, which may be in the form of flakes, powder, pellets or the like or consolidated or pre-molded forms in layers. This may be done such that there can be distinct regions in the consolidated form containing different concentrations of additives such as antioxidant(s), therapeutic agent(s) and/or crosslinking agent(s). Layering can be done any method that deposits desired polymeric material in desired locations. These methods may include pouring, scooping, painting, brushing spraying. This deposition can be aided by materials, templates and such supporting equipment that do not become an eventual part of the consolidated polymeric material. Whenever a layered-molded polymeric material is described and is used in any of the embodiments, it can be fabricated by: (a) layered molding of polymeric resin powder or blends of polymeric material containing a specific additive(s) where one or more layers contain said additive and one or more layers do not contain said additive(s); (b) molding together of layers of polymeric material containing different or identical concentration of additives such as therapeutic agent(s), antioxidant(s) and/or crosslinking agent(s).

Layering and spatial control of additive concentrations and polymeric material morphology are described in WO2008/092047A1, U.S. Pat. Nos. 9,968,709, 9,433,705, and U.S. Pat. No. 8,569,395 (Muratoglu et al.), which are incorporated by reference in their entireties.

One or more of the layers can be treated before or during molding by heating, or high temperature melting. Methods of high temperature melting are described in U.S. Pat. No. 9,731,047, WO2010/096771A2, U.S. Pat. No. 8,933,145 (Oral et al.), which are incorporated by reference in their entireties.

The layer or layers to be molded can be heated in liquid(s), in water, in air, in inert gas, in supercritical fluid(s) or in any environment containing a mixture of gases, liquids or supercritical fluids before pressurization. The layer or layers can be pressurized individually at room temperature or at an elevated temperature below the melting point or above the melting point before being molded together. The temperature at which the layer or layers are pre-heated can be the same or different from the molding or dwell temperature(s). The temperature can be gradually increased from pre-heat to mold temperature with or without pressure. The pressure to which the layers are exposed before molding can be gradually increased or increased and maintained at the same level.

During consolidation, different regions of the mold can be heated to different temperatures. The temperature and pressure can be maintained during molding for 1 second up to 1000 hours or longer. During cool-down under pressure, the pressure can be maintained at the molding pressure or increased or decreased. The cooling rate can be, for example, 0.0001° C./minute to 120° C./minute or higher, or any value therebetween. Cooling can be done at any rate. The cooling rate can be different for different regions of the mold. After cooling down to about room temperature, the mold can be kept under pressure for 1 second to 1000 hours, or any value therebetween. Or the pressure can be released partially or completely at an elevated temperature.

The term “heating” refers to bringing a material to a temperature, generally a temperature above that of its current state. It can also refer to maintaining said temperature for a period of time, that is, in some instances it can be used interchangeably with ‘annealing’. Heating can be done at any rate. The heating rate can be, for example, from 0.001° C./min to 1000° C./min, or any value therebetween, or it can be between 0.1° C./min to 100° C./min, or it can be from 0.5° C./min to 10° C./min, or it can be any rate from 1° C./min to 50° C./min in 1° C. intervals. The heating can be done for any duration. Heating time can be from 0.1 minutes to 100 years or from 1 minute to 24 hours or from 1 minute to 12 hours, or 30 minutes to 10 hours, or 5 hours, or 6 hours, or 8 hours, or any value therebetween.

The term “cooling” refers to bringing a material to a temperature, generally a temperature below that of its current state. It can also refer to maintaining said temperature for a period of time, that is, in some instances it can be used interchangeably with ‘annealing’. Cooling can be done at any rate. The cooling rate can be from 0.001° C./min to 1000° C./min, or it can be between 0.1° C./min to 100° C./min, or it can be from 0.5° C./min to 10° C./min, or it can be any rate from 1° C./min to 50° C./min in 1° C. intervals, or 2.5° C./min, or any value therebetween. The cooling can be done for any duration. Cooling time can be from 0.1 minutes to 100 years or from 1 minute to 24 hours or from 1 minute to 12 hours, or 30 minutes to 10 hours, or 1 hour, or 2 hours, or 5 hours, or 6 hours, or 8 hours, or any value therebetween.

The terms “about” or “approximately” in the context of numerical values and ranges refers to values or ranges that approximate or are close to the recited values or ranges such that the invention can perform as intended, such as utilizing a method parameter (e.g., time, dose, dose rate/level, and temperature), having a desired amount of antibiotics, desired degree of cross-linking and/or a desired lack of or quenching of free radicals, as is apparent to the skilled person from the teachings contained herein. This is due, at least in part, to the varying properties of polymer compositions. Thus, these terms encompass values beyond those resulting from systematic and random error. These terms make explicit what is implicit, as known to the person skilled in the art.

The term “sterile” refers to a condition of an object, for example, an interface or a hybrid material or a medical implant containing interface(s), wherein the interface is sufficiently sterile to be medically acceptable, i.e., will not cause an infection or require revision surgery. The object, for example a medical implant, can be sterilized using ionizing radiation or gas sterilization techniques. Gamma sterilization is well known in the art. Electron beam sterilization is also used. Ethylene oxide gas sterilization and gas plasma sterilization are also used. Autoclaving is another method of sterilizing medical implants. Exposure to solvents or supercritical fluids for sufficient to kill infection-causing microorganisms and/or their spores can be a method of sterilizing.

The term ‘wear’ refers to the removal of material from the polymeric material during articulation or rubbing against another material. For UHMWPE, wear is generally assessed gravimetrically after an initial creep deformation allowance in number of cycles of motion. The term ‘wear resistant’ refers to the state of a polymeric material where it has low wear. For example, the wear rate is tested on cylindrical pins (diameter 9 mm, length 13 mm) on a bidirectional pin-on-disc wear tester in undiluted bovine calf serum at 2 Hz in a rectangular pattern (5 mm×10 mm) under variable load with a maximum of 440 lbs. as described in Bragdon et al. (J Arthroplasty 16: 658-665 (2001)). Initially, the pins are subjected to 0.5 million cycles (MC), after which they are tested to 1.25 million cycles with gravimetric measurements approximately every 0.125 MC. The wear rate is determined by the linear regression of the weight loss as a function of number of cycles from 0.5 to 1.25 MC.

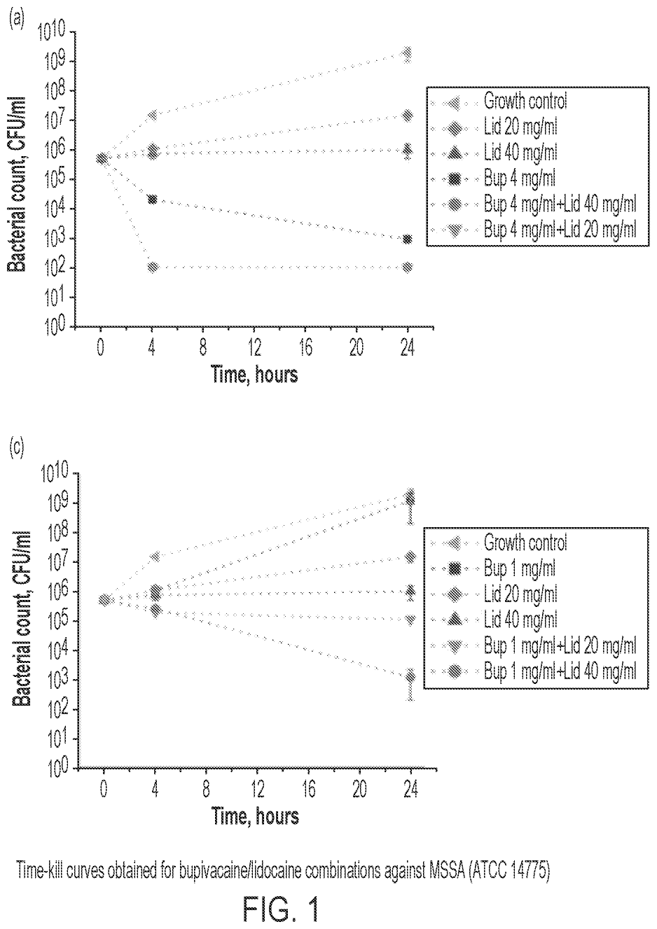

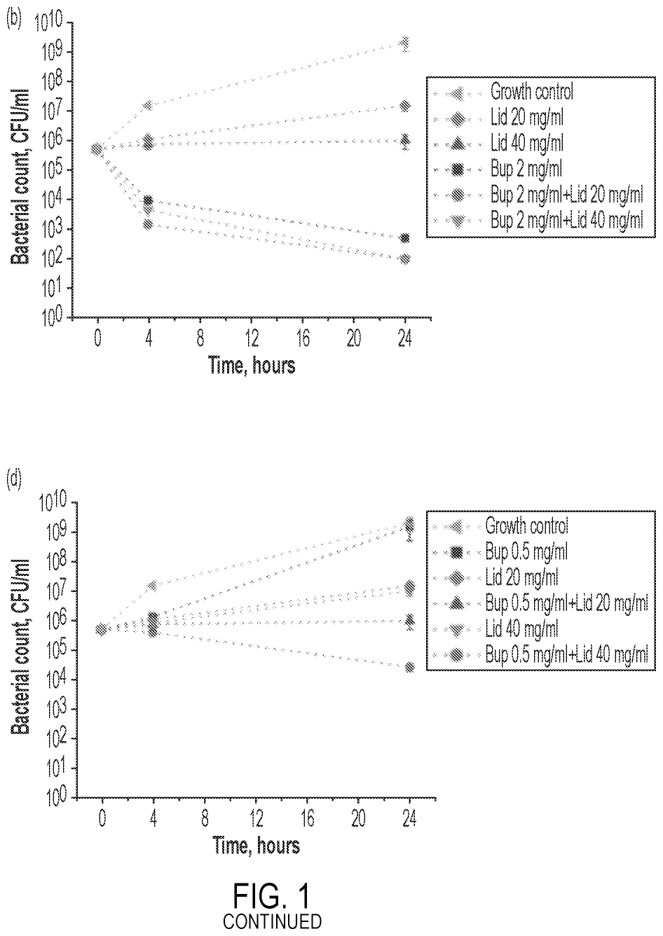

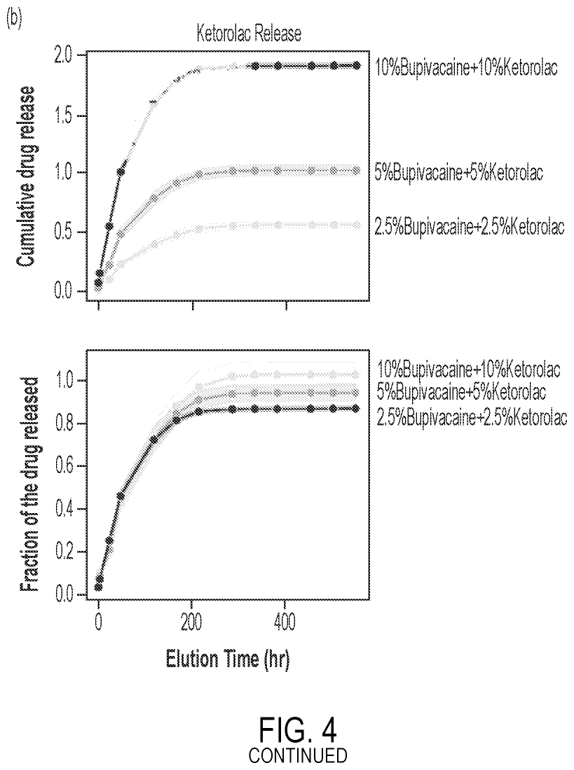

The term “surface” refers to any part of the outside of a solid-form material, which can be exposed to the surrounding liquid, gaseous, vacuum or supercritical medium. The surface can have a depth into the bulk of the material (normal to the surface planes), from several microns (μm) to several millimeters. For example, when a ‘surface layer’ is defined, the layer can have a thickness of several nanometers to several microns (μm) to several millimeters. For example, the surface layer can be 100 microns (100 μm) or 500 microns (500 μm) or 1000 microns (1 mm) or 2 mm or it can be between 2 and 5 mm, or any value therebetween. The surface or surfaces can also be defined along the surface planes. For example, a 5 mm wide and 15 mm long oval section of the articulating surface of a tibial knee insert can be defined as a ‘surface’ to be layered with a UHMWPE containing additives. These surfaces can be defined in any shape or size and the definition can be changed at different processing step. (FIGS. 12-1 and 13-1)

The term “packaging” refers to the container or containers in which a medical device is packaged and/or shipped. Packaging can include several levels of materials, including bags, blister packs, heat-shrink packaging, boxes, ampoules, bottles, tubes, trays, or the like or a combination thereof. A single component may be shipped in several individual types of package, for example, the component can be placed in a bag, which in turn is placed in a tray, which in turn is placed in a box. The whole assembly can be sterilized and shipped. The packaging materials include, but are not limited to, vegetable parchments, multi-layer polyethylene, Nylon 6, polyethylene terephthalate (PET), and polyvinyl chloride-vinyl acetate copolymer films, polypropylene, polystyrene, and ethylene-vinyl acetate (EVA) copolymers.

### Hydrogel Polymeric Material

As used herein, the term “monomer” refers to a molecule that may bind covalently to other molecules to form a polymer. The process by which the monomers are combined to form a polymer is called polymerization. Common monomers useful in the methods described herein include, but are not limited to, ethylene oxide, DL-lactide, glycolide, and ε-caprolactone.

As used herein, the term “polymer segment” means and includes a grouping of multiple monomer units of the same type (i.e. a homopolymer segment) or of different types (i.e. a co-polymer segment) of constitutional units joined together into a continuous polymer chain.

As used herein, the term “polymer block” means and includes a grouping of multiple monomer units of the same type (i.e. a homopolymer block) or of different types (i.e. a co-polymer block) of constitutional units joined together into a continuous polymer chain that forms part of a larger polymer of even greater length.

As used herein, the term “block co-polymer” means and includes a polymer composed of chains where each chain is composed of two or more polymer blocks as defined above. A block co-polymer may be represented herein by (Aₙ-Bₘ), where A and B represent monomers and n and m each represent the number of repeats.

As used herein, the term “random co-polymer” means and includes a polymer chain formed from two different monomers arranged in a pattern having no particular order to form a polymer segment. Random co-polymers may be represented by (An-r-Cp), where the capital letters A and C represent monomers, n and p each represent the number of repeats, and r represents that the sequence of A and C monomers is random and has no particular order.

As used herein, the term “chemical moiety” represents a grouping of atoms in a specific arrangement which form covalent chemical bonds in a specific sequence and type.

By “connecting moiety” what is meant is a molecule or part of molecule that connects biodegradable moiety with biodegradable moiety, biodegradable moiety with cross-linkable moiety, and/or cross-linkable moiety with cross-linkable moiety. Such connecting moiety(ies) can be chosen from the group of but are not limited to polyethylene glycol, polyethylene oxide, polypropylene glycol, 1,6-hexanediol, 2,2,6,6-Tetrakis(hydroxymethyl)cyclohexanol, ethylene glycol, cyanuric acid. Such connecting moieties consist of a mixture of one or more types and consists of a mixture of different molecular weight distributions. In a preferred embodiment, the connecting moiety is liquid at room temperature. In one embodiment, the connecting moiety can be a mixture of polyethylene glycol and propylene glycol. In another embodiment, the connecting moiety can be a mixture of polyethylene glycol with average molecular weight of 200 and polyethylene glycol with average molecular weight of 400. In another embodiment, the connecting moiety has a random distribution(s) of weight average molecular weight polyethylene glycol. In the preferred embodiment, the connecting moiety can be polyethylene glycol with weight average molecular weight of 200 (PEG 200). In another preferred embodiment, the connecting moiety can be polyethylene glycol with weight average molecular weight of 400 (PEG 400).

By “biodegradable moiety”, what is meant is a molecule or part of molecule that can be degraded (e.g. cleaved and/or destroyed and/or decomposed inside the body) and eliminated by the body. The cleaving, destroying, or decomposing can be through hydrolysis, enzymatic degradation, modification by the liver, excretion by the kidney(s) and/or combinations thereof. Modification by the liver means the changing of the degraded polymer by the liver. Such biodegradable moiety can be but not limited to poly(lactide) (PLA), poly(glycolide) (PGA), poly(epsilon-caprolactone) (PCA), poly(dioxane) (PDA), poly(trimethylene carbonate) (PTMC), and combinations thereof. In one embodiment, the biodegradable moiety is polyglycolide. In another embodiment, the biodegradable moiety is polylactide-co-polyglycolide. In another embodiment, the biodegradable moiety is polytrimethylene carbonate-co-poly(epsilon-caprolactone). In a preferred embodiment, the biodegradable moiety is polylactide with length of 1-8 lactoyl groups. In another preferred embodiment, the biodegradable moiety is polyglycolide with length of 1-8 glycolyl groups. In another preferred embodiment, the biodegradable moiety is polycaprolactone with length of 1-8 epsilon-caprolactone groups. In certain preferred embodiments, the biodegradable moiety is a polylactide with 2-4 lactoyl groups.

By “cross-linkable moiety”, what is meant is a molecule or part of a molecule that can form one or more new bond(s) (covalent and/or non-covalent) with another molecule, preferably a macromonomer to create a network of molecule(s) and/or macromonomers. Such cross-linkable moieties can comprise acrylate(s), methacrylate(s), thiols, carboxyls, hydroxyls, amino groups, isocyanates, azides, isothiocyanates, epoxides, and/or combinations thereof). In a preferred embodiment, the cross-linkable moiety comprises acrylate(s), methacrylate(s), or combinations thereof. In more preferred embodiment, the cross-linkable moiety comprises an acrylate group As used herein, the term “photoinitiator” represents a chemical compound that can produce radical species and/or promote radical reactions when exposed to light irradiation. Common photoinitiators useful in the methods, compositions, and systems described herein include, but are not limited to, benzoin ethers, benzyl ketals, α-dialkoxyacetophenones, α-hydroxyalkylphenones, α-amino alkylphenonones, acylphophine oxides, peroxides, and acylphosphinates, azobisisobutyronitrile, 1,1′-azobis(cyclohexanecarbonitrile), di-tert-butyl peroxide, benzoyl peroxide, methyl ethyl ketone peroxide, and acetone peroxide. An exemplary photoinitiator is phenylbis(2,4,6-trimethylbenzoyl) phosphine oxide.

As used herein, the term “macromer” represents a polymer chain containing a chemical moiety at one or both ends of the polymer chain which can undergo further chemical reactions to either extend the polymer chain or form a network of polymer chains.

As used herein, the term “gel” or “gel network” represents a non-fluid network of polymer chains formed from a previously fluid solution of polymer chains and possibly including other additives. A gel may be formed by a network of polymer chains joined through covalent bonds, nonlinear polymerization, or through non-covalent aggregation of polymer chains or segments. The process by which a fluid solution of polymer chains and possibly including other additives are combined to form a gel network is called gelation. By way of non-limiting example, gelation may be caused by a chemical reaction initiated by light, heat, change of temperature, or other radiation. Optionally, a gel may have its volume expanded by a fluid or solvent, e.g. water.

As used herein, the term “degradable” or “degradable material” means that the material decomposes through either physical means or chemical means or both physical and chemical means at a certain period of time after the material is implanted as a medical device. By “biodegradation” it is meant to include cleaving, destroying, or decomposing through hydrolysis, enzymatic degradation, biological modification by the liver, excretion by the kidney(s) and combinations of these modes of degradation. Biological modification by the liver means the changing of the chemical structure of the degraded polymer by the liver. As a result, the drug eluting polymer disappears in a certain period after implantation and therefore is no longer a potential surface for colonization by bacteria. The time that it takes for the material to degrade may be as short as one minute or as long as ten years or any length of time between one minute and ten years. The material degradation may be measured by a loss of mass of material, loss of volume of material, decrease in the mechanical stiffness of the material, or change in the molecular structure of the material.

In the representations used herein, (e.g. (Aₙ), B, (Aₙ-Bₘ-Aₙ), (R-Aₙ-Bₘ-Aₙ-R), ((Aₙ-r-Cₚ)-Bₘ-Aₙ-(Aₙ-r-Cₚ)), and (R-(Aₙ-r-Cₚ)-Bₘ-Aₙ-(Aₙ-r-Cₚ)-R)), the capital letters A, B, and C represent monomers. Subscript lowercase letters immediately adjacent to a capital letter indicates the number of repeats of the monomer of that type in a sequence. The capital letter R represents a chemical moiety. Segments or blocks of monomers are set apart by parentheses. Lowercase letters between capital letters and set apart by hyphens indicate an ordering arrangement between the monomers in the sequence. Specifically, “r” indicates that the ordering is random. Hyphens setting apart polymer segments or polymer blocks or chemical moieties indicate that the adjoining polymer segments or blocks or chemical moieties are connected in sequence.

### SUMMARY OF THE INVENTION

The present invention relates to methods of making therapeutic medical implants with optimum properties for clinical performance. We propose to use drug-loaded polymeric materials, such as UHMWPE and degradable hydrogels, to provide post-surgical antibacterial prophylaxis. The invention relates to methods of making polymeric materials loaded with two drugs that provide synergistic antibacterial effect. Alternatively, the invention also relates to methods of making layered constructs of polymeric material consisting of two or more therapeutic agents with synergistic antibacterial effect, so that mechanical properties are not compromised. Alternatively, the invention relates to methods of making medical implants with non-homogenous distribution of two of more therapeutic agents with synergistic antibacterial effect.

Various embodiments of the invention are described below. These descriptions are meant to be permissive, and do not restrict the invention in any way or manner.

In the present invention, methods of making polymeric materials containing two or more drugs with synergistic antibacterial activity are described such that these materials can be used in conjunction with bone cement/antibiotic-eluting bone cement as a fixation aid.

In some embodiments, a drug-eluting polymeric material made by the methods described herein can be made in the shape of a bearing surface, for example an acetabular liner. In this application, the liner can be made of UHMWPE containing two or more drugs with synergistic antibacterial activity and can be used during primary arthroplasty. This drug-eluting liner can provide effective antibacterial prophylaxis, substantially reducing post-arthroplasty complications.

Alternatively, a drug-eluting polymeric material made by the methods described herein can be made in the shape of small plugs made of UHMWPE containing two or more drugs with synergistic antibacterial activity and can be used pressfit into an acetabular shell. Commonly, some metallic components that come into contact with bony surfaces can have holes to accommodate screws for additional fixation strength and stability if desired. Many of these screw holes do not end up accommodating screws during the operation and are left ‘as is’. One or more drug-eluting polymeric materials made by the methods described herein can be placed into these holes instead of screws.

Alternatively, a drug-eluting polymeric material made by methods described herein can be made the shape of small plugs made of UHMWPE containing two or more drugs with synergistic antibacterial activity and can be used pressfit into the bearing surface. For example, a tibial insert, which is the polymeric bearing surface used in a total knee replacement, can be made with small indentations on its backside (which would typically be in direct contact with the tibial base tray or be cemented into the tibial plateau) into which said small plugs can fit. The bearing surface can then be fit with the drug-eluting plug(s) and placed into a tibial base tray (which comes into contact with the bony surfaces on the tibial side of the implant with or without bone cement as a fixation aid).

In some embodiments, the degradable hydrogel containing two or more drugs with synergistic antibacterial activity can be applied on a substrate material or device that is implanted during arthroplasty. The substrate material may be any material, for example metal, ceramic, polymeric material. The material may be porous, contain holes, or other such geometric features. In some embodiments the degradable hydrogel may fill or partially fill the holes or pores of the substrate material to provide even higher drug release, and thus even more efficient antibacterial prophylaxis.

In some embodiments, the degradable hydrogel containing two or more drugs with synergistic antibacterial activity can be spread as a thin layer on top of a substrate material or device that is implanted during arthroplasty. The substrate material may be any material, for example metal, ceramic, polymeric material. The material may be porous, contain holes, or other such geometric features. In some embodiments the degradable hydrogel may fill or partially rill the holes or pores of the substrate material to provide even higher drug release, and thus even more efficient antibacterial prophylaxis.

In some embodiments, the degradable hydrogel containing two or more drugs with synergistic antibacterial activity may physically cut, re-shaped, or machined to produce a solid gel with a different two-dimensional or three-dimensional shape. Other geometric features may be created as the result of cutting or re-shaping or machining, including but not limited to holes, indentations, tapered holes, blunt holes, or screw holes.

In an embodiment, the invention provides a method of making an antibacterial medical implant wherein the method comprises: (i) Providing a first polymeric material; (ii) Providing two or more therapeutic agents with synergistic antibacterial activity; (iii) Blending the polymeric material with the therapeutic agents, thereby making a therapeutic agent-blended polymeric material; (iv) Processing the therapeutic agent-blended polymeric material, thereby making a processed antibacterial, polymeric material; (v) Fashioning a medical implant from the processed antibacterial, polymeric material, thereby making an antibacterial medical implant.

In an embodiment, the invention provides a method of making an antibacterial polymeric material wherein the method comprises: (i) Providing a first polymeric material, (ii) Providing two or more therapeutic agents with synergistic antibacterial activity; (iii) Blending the polymeric material with two or more therapeutic agents, thereby making a therapeutic agent-blended polymeric material; (iv) Processing the therapeutic agent-blended polymeric material, thereby making a processed antibacterial, polymeric material.

In an embodiment, a method of making a layered consolidated material wherein the method comprises: (i) Providing a polymeric material; (ii) Blending the polymeric material with two or more therapeutic agents with synergistic antibacterial activity; (iii) Providing a second polymeric material; (iv) Blending the second polymeric material with a cross-linking agent; (v) Layering the drug-blended polymeric material and the cross-linking agent-blended second polymeric material; (vi) Consolidating the layered polymeric materials; thereby obtaining a cross-linked implant with antibiotic-rich regions. In this embodiment, the polymeric material in the first and second layers can be the same or different or a mixture containing different polymers. The said medical implant can be implanted abutting another surface such as a porous or non-porous metal.

In an embodiment, a method of making medical implant wherein the method comprises: (i) Providing a polymeric material; (ii) Blending the polymeric material with at least two different drugs, which can include a sodium-channel blocker, an NSAID, an antibiotic, an antioxidant, that possess synergistic antibacterial effect; (iii) Layering the multiple drug-blended polymeric material polymeric and the polymeric material without drugs; (iv) Consolidating the layered polymeric materials; thereby obtaining an implant with drug-rich regions; (v) Exposing at least parts of the implant to radiation, thereby obtaining a cross-linked therapeutic medical implant. In embodiments where irradiation or cross-linking is described, the regions or layers of the polymeric material, where the drugs are located, can coincide with crosslinked regions or are different from the crosslinked regions. Cross-linking of different regions can be done by such methods as spatially controlling radiation energy absorbed by regions of the polymeric material, or by having different concentration of crosslinking or anticross-linking agents. Controlling radiation exposure by using shields or changing the energy of the radiation or selectively irradiating part of a medical device are described in U.S. Pat. No. 7,381,752 (Muratoglu) and 10,000,305 (Oral).

In an embodiment, a method of making a degradable, additive-blended polymeric material wherein the method comprises: (i) Providing liquid polymerizable macromer composed of a cross-linkable moiety ((Bₘ-Aₙ-Bₘ-R) or (R-Bₘ-Aₙ-Bₘ-R)); (ii) Blending the liquid, polymerizable mixture with two therapeutic agents (Drug X and Drug Y) with synergistic antibacterial activity; (iii) Exposing the additive-blended, liquid polymerizable macromer(s) to as external stimulus which can create free radicals for a period of time, thereby forming a degradable, additive-blended gel. Herein, drug X can be a sodium-channel blocker, or antibiotic, or anesthetic, or antioxidant, or NSAID. Drug Y can be sodium-channel blocker, or antibiotic, or anesthetic, or antioxidant, or NSAID. Methods of making degradable, polymeric material and liquid polymerizable macromer are described in PCT/US2017/016506, and U.S. Provisional Application No. 62/969,247, which are incorporated here in their entirety.

In an embodiment, a method of making a degradable, additive-blended medical implant wherein the method comprises: (i) Providing liquid polymerizable macromer composed of a cross-linkable moiety ((Bₘ-Aₙ-Bₘ-R) or (R-Bₘ-Aₙ-Bₘ-R)); (ii) Blending the liquid, polymerizable mixture with two therapeutic agents (Drug X and Drug Y) with synergistic antibacterial activity; (iii) Exposing the additive-blended, liquid polymerizable macromer(s) to as external stimulus which can create free radicals for a period of time, thereby forming a medical implant. Herein, drug X can be sodium-channel blocker, or antibiotic, or anesthetic, or antioxidant, or NSAID. Drug Y can be sodium-channel blocker, or antibiotic, or anesthetic, or antioxidant, or NSAID. In this embodiment, a medical implant can be performed in a sterile environment or the medical implant can be terminally sterilized before implantation.

In any embodiment, the following drug types can be blended with polymeric materials: analgesics, anesthetics, sodium-channel blockers, NSAIDs, antioxidants, antibiotics. Drugs of the same type can also be blended with the polymeric material. Drug combinations chosen to be blended with the polymeric material should exhibit antibacterial synergistic or additive effects. For example, lidocaine and bupivacaine can be blended with the polymeric material to provide synergistic antibacterial effect. Alternatively, gentamicin and ketorolac can be blended with the polymeric material to provide synergistic antibacterial effect. Alternatively, gentamicin and prilocaine can be blended with the polymeric material to provide additive antibacterial effect.

In any of the embodiments, the concentration of drugs in the polymeric material can be 0.001 wt % to 99 wt %, or any value therebetween, preferably above 5 wt % to 25 wt %, more preferably from 6 wt % to 10 wt %. When multiple drugs are used, their concentrations can be the same or different. The ratio of drug concentrations should be chosen such that they provide synergistic antibacterial effect. For example, 4 wt % of Bupivacaine, and 20 wt % of Lidocaine can be loaded in UHMWPE, and upon release provide synergistic antibacterial activity.

In any of the embodiments, the provided polymeric material can be pre-mixed with other additives that are not intended to provide therapeutic effects. For example, the provided polymeric material can be UHMWPE pre-mixed or pre-blended with 0.2 wt % antioxidant to protect the polymeric material against oxidation. In any of the embodiments, the polymeric material provided to be blended with therapeutic agents can be pre-mixed or pre-blended with other additives. In any of the embodiments, the polymeric material provided to be blended with therapeutic agents can contain additives at the same or different concentration compared to the polymeric material provided to be used without blending with therapeutic agents.

In any of the embodiments, the layering can be done such that only the desired parts of an implant contain the therapeutic agents. For example, the rim of an acetabular cup can contain therapeutic agents, whereas the articular surface on the inside of the cup can be made from polymeric material. Alternatively, the articular surface of a tibial insert or just condylar regions can contain therapeutic agents. Alternatively, the side surfaces or the backside surface of a tibial insert can contain therapeutic agents. Layering can be done by methods such as spraying of the polymeric material blended with therapeutic agents such that the location of the polymeric material containing therapeutic agents can be controlled.

In any of the embodiments, as desired, the polymeric material or the consolidated polymeric material or medical implants or medical implant preforms can be heated before or after any step. The heating can serve to diffuse components, aid in the mixing of components, or relieve stresses. In some embodiments the polymeric material or the preform or the implant is heated prior to and/or after radiation crosslinking or crosslinking by chemical methods such as peroxides. In some embodiments, the polymeric material is heated before, during or after blending or diffusing with additives.

In any of the embodiments, as desired, the therapeutic agents can be incorporated into the polymeric material by diffusion. For example, a therapeutic agent can be contacted with a polymeric material or consolidated polymeric material or a crosslinked consolidated material to diffuse the therapeutic agent into the surface(s) of the polymeric material. The therapeutic agent can be contacted in pure form, in a gas, in solution, in emulsion, slurry, or in a supercritical fluid. In any of the embodiments, as desired, the therapeutic agents can be processed before incorporating into the polymeric material. For example, the therapeutic agent can be maintained under an environment with a predetermined amount of oxygen, in vacuum, in dry or hydrated or emulsified form, mixed with at least one other therapeutic agent. The therapeutic agent can be maintained heated or cooled in contact with environments containing different levels of humidity.

In any of the embodiments, as desired, the different layers of polymeric material have different concentrations of the same or different therapeutic agent(s). In certain embodiments, one of the different layers of polymeric material has no added therapeutic agent.

In an embodiment, the consolidated polymeric material is irradiated using ionizing radiation such as gamma, electron-beam, or x-ray to a dose level between about 1 and about 10,000 kGy, or any value therebetween, preferably about 25 to about 250 kGy, preferably about 50 to about 150 kGy, preferably about 65 kGy, preferably about 85 kGy, preferably about 100 kGy, or preferably about 120 kGy.

In some embodiments, the medical implants described are cleaned before packaging and sterilization. Sterilization can be performed by chemical methods such as ethylene oxide sterilization or by radiation methods such as gamma or electron beam irradiation.

In any of the embodiments, a therapeutic medical implant or a therapeutic polymeric material can be used in conjunction with other methods in the field of application. For example, in total hip replacement, a therapeutic medical implant can be in the shape of an acetabular liner to be used as an articular surface. Or a therapeutic medical implant can be in the shape of small plugs or ‘manhole covers’ that can be placed in the existing screw holes of the acetabular shell.

### EXAMPLES

### Example 1. Assessment of Antibacterial Activity of Single Drugs

Antibacterial activity of sodium channel blockers (lidocaine, bupivacaine, tetracaine, procaine, prilocaine), NSAIDs (ketorolac and tolfenamic acid), antioxidants (resveratrol and curcumin), and antibiotics (gentamicin and vancomycin) was performed against methicillin-sensitive Staphylococcus aureus (MSSA, ATCC 12600). The bacterial susceptibility tests were conducted as outlined in the Clinical and Laboratory Standard Institute (CLSI) protocol M07-A10. Bacterial suspensions with a concentration of approximately 5*10⁵ CFU/ml were prepared in Lysogeny broth and mixed with the tested drugs. Following 24-hour incubation at 37° C., turbidity of the resulting suspensions was visually evaluated, and the minimum inhibitory concentrations (MIC) was determined as the lowest concentration of a single drug that inhibits bacteria proliferation (clear solution). Minimum bactericidal concentration (MBC) was determined as the minimum concentration of the tested drug needed to kill 99.9% of bacteria (or 3−log 10 reduction). The obtained results suggested that tested analgesics possessed moderate antimicrobial properties against MSSA. The MIC values are presented in Table 1. The MBC was measured for lidocaine, bupivacaine, ketorolac, and gentamicin. The MBC of lidocaine and gentamicin were found to be 128 mg/ml and 2 μg/ml, respectively. The MBC of bupivacaine and ketorolac were not determined, as these concentrations were higher than the drug solubility limits in growth medium, which are approximately 15 mg/ml and 18 mg/ml, respectively.

### Example 2. Assessment of the Combinational Antibacterial Effects

Combined antibacterial effects of lidocaine, bupivacaine, prilocaine, procaine, tetracaine, tolfenamic acid ketorolac, curcumin, resveratrol, gentamicin, and vancomycin was evaluated using the checkerboard test against two MSSA strains (ATCC® 12600 and 14775), and S. epidermidis strain (ATCC 12228). Drug combinations with various ratios were prepared and mixed with approximately 5*10⁵ CFU/ml of MSSA in Lysogeny broth. Following 24-hour incubation at 37° C. in a 96 well-plate, the turbidity of the resulting suspension was visually observed, and synergistic ratios were determined. The fractional inhibitory concentration indices (ΣFIC) were calculated. Drug combinations were considered to be synergistic if ΣFIC≤0.5; an additive/indifference effect was observed if 0.5<ΣFIC<4; and ΣFIC>4 represented antagonism. The results are presented in tables 2, 3, and 4.

### Example 3. Time-Kill Experiments

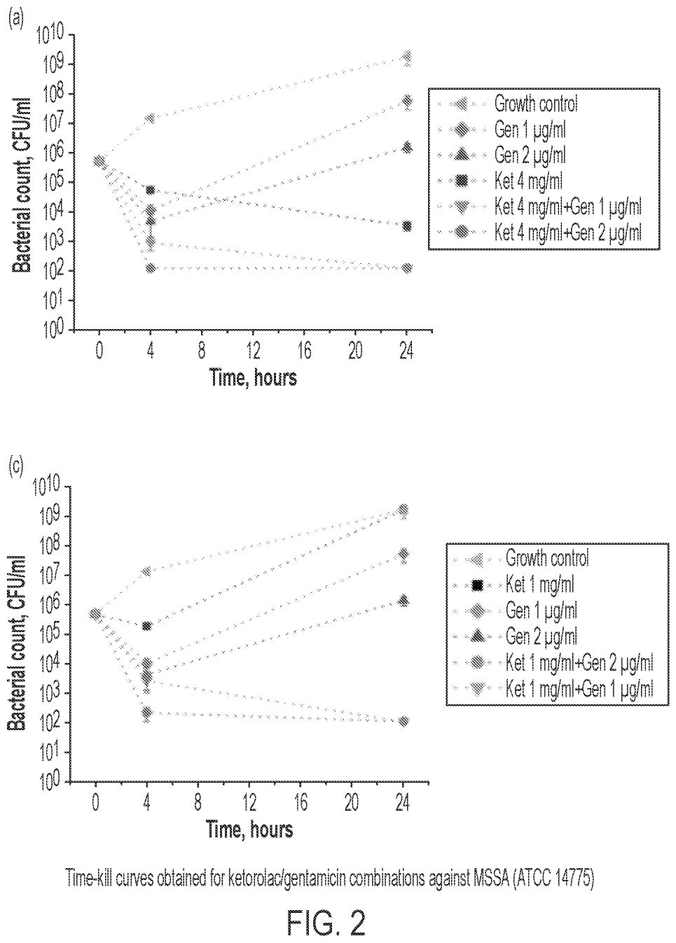

Time-kill curves employing 3 time-points (0, 4, 24 hours) over the period of 24 hours were obtained for characterization of the antibacterial activity of selected combinations against MSSA (ATCC® 14775). Tested combinations of drugs were incubated with 5*10⁵ CFU/ml of bacteria in Lysogeny broth at 37° C. At a given time-point, aliquots were collected, and bacterial concentrations were determined using the spread-plate method. The minimum accurately countable concentration was 10² CFU/ml. A combination was defined to be synergistic if 2−log 10 kill between the combination, and its most active constituent was observed after 24 hours. 1− to 2−log 10 reduction in bacteria when comparing a combination with the most active single agent was an evidence of improved activity. The increase of bacterial concentration represented antagonism. FIG. 1 shows that when used concurrently, 4 mg/ml of bupivacaine and 20 mg/ml of lidocaine showed pronounced synergistic effect at 4 h. At 24 h synergistic kill was achieved if bupivacaine at 1 mg/ml and lidocaine at 40 mg/ml were used. Other combinations of bupivacaine and lidocaine showed 1− to 2−log 10 kill, indicating improved antibacterial activity. Time-kill curves obtained for the ketorolac/gentamicin combinations (FIG. 2) were in good agreement with the checkboard test showing distinct synergism at 4 h when 2 mg/ml of ketorolac and 1 μg/ml of gentamicin were used concurrently. At lower concentrations, this drug combination also yielded synergistic effect at 24 h. Improved antibacterial activity was evident for all combinations at the 4 h time point. The obtained time-kill curves showed that gentamicin monotherapy regimens yielded a regrowth phase between 4 and 24 h, while there was no regrowth with ketorolac/gentamicin combinations. The combination regimens were significantly more active over the first 4 h showing substantially higher initial kill when compared with monotherapy. Similar effects were observed for bupivacaine/lidocaine combinations.

### Example 4. Preparation of Dual Drug-Loaded UHMWPE

Drugs were mixed at a desired ratio, passed through a 75 μm sieve, and then blended with UHMWPE resin (GUR 1020). Dual drug-loaded UHMWPE blocks were prepared using compression molding in a custom mold and molded for 54 minutes at 170° C. under a pressure of 25 MPa.

### Example 5. Drug Release from Dual Drug-Loaded UHMWPE

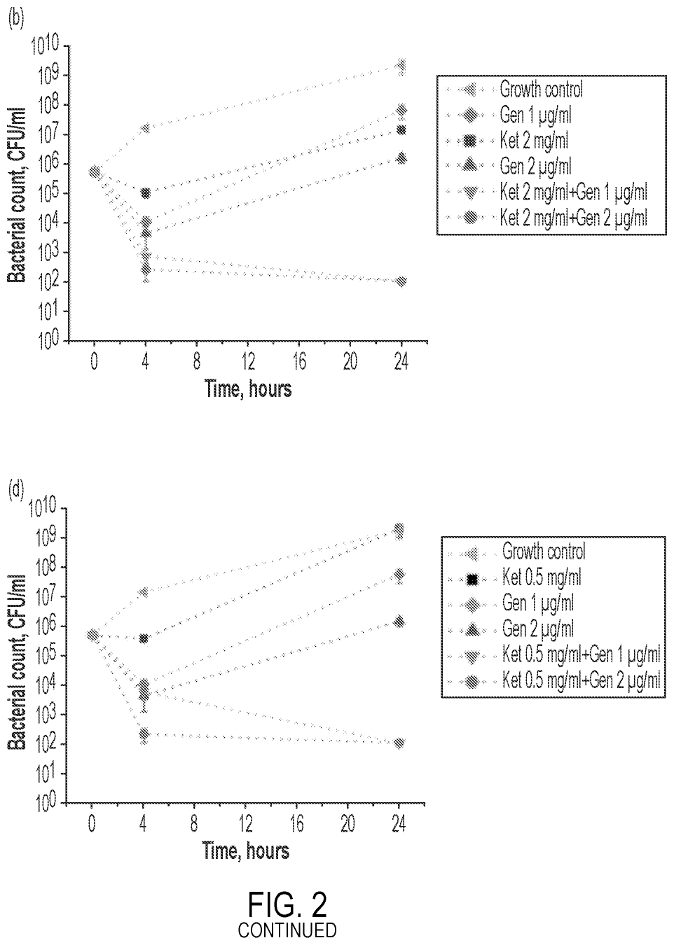

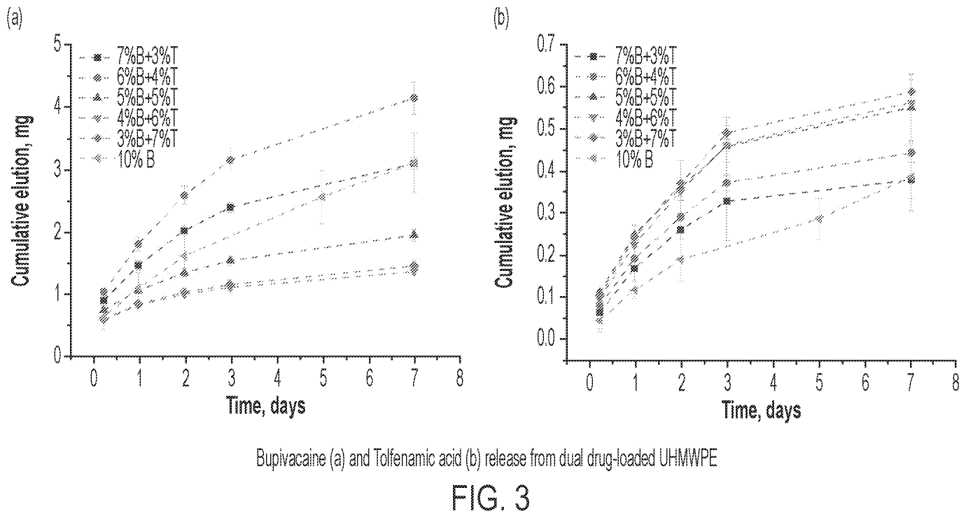

Drugs were mixed at a desired ratio, passed through a 75 μm sieve, and then blended with UHMWPE resin (GUR 1020). Dual drug-loaded UHMWPE blocks were prepared using phase-separation compression molding in a custom mold and molded for 54 minutes at 170° C. under a pressure of 25 MPa. The following formulations were prepared: 10 wt % Bupivacaine-loaded UHMWPE, 6 wt % Bupivacaine/4 wt % Tolfenamic acid-loaded UHMWPE, 5 wt % Bupivacaine/5 wt % Tolfenamic acid-loaded UHMWPE, 4 wt % Bupivacaine/6 wt % Tolfenamic acid-loaded UHMWPE, 3 wt % Bupivacaine/7 wt % Tolfenamic acid-loaded UHMWPE, 7 wt % Bupivacaine/3 wt % Tolfenamic acid-loaded UHMWPE, 10 wt % Tolfenamic acid-loaded UHMWPE. The obtained materials were cut into 3×5×20 mm strips, transferred to 1.7 ml of PBS, and incubated in syringes under mild shaking (100 rpm) at room temperature. At a given time point, the release medium was collected, the syringes were rinsed, and the medium was replaced. High-performance liquid chromatography (HPLC) was used to measure drug concentration in the eluent. Drug concentration was measured using a Waters Alliance 2695 separations module (Milford, Mass.) and a Waters 2487 UV detector at a detection wavelength of 210 nm. A Waters Nova-Pak C18 column with 4 μm particle size, 3.9 mm diameter and 150 mm length was used. An isocratic mixture of 50% acetonitrile and 50% deionized water with 0.2% phosphoric acid was used as a mobile phase. The flow rate was 1.0 ml/min, and the sample injection volume was 5 μl. 6 replicates were performed to obtain drug release kinetics. The results are shown in FIG. 3.

### Example 6. Synthesis of MA-PLA₄-PEG₉-PLA₄-MA

2.51 g of PEG with a molecular weight of 400 g/mol was mixed with 3.60 g of DL lactide and 55 mg of stannous octoate. Then the mixture was heated in a microwave for 2 minutes to obtain PLA₄-PEG₉-PLA₄. Methacrylic anhydride (2.59 g) was added and the obtain mixture was heated in a microwave for 2 minutes to synthesize MA-PLA₄-PEG₉-PLA₄-MA.

### Example 7. Incorporation of Drugs into MA-PLA₄-PEG₉-PLA₄-MA

A photoinitiator solution (10 wt % Phenylbis(2,4,6-trimethylbenzoyl)phosphine oxide in acetone) was added to the macromer (MA-PLA₄-PEG₉-PLA₄-MA) at a ratio of 50 μl solution:1 g macromer. Two different drugs (ketorolac and bupivacaine) were incorporated into the macromer via manual stirring. The macromer/drug mixture was then injected into a mold and irradiated with ultraviolet light with a wavelength of 365 nm for 5 minutes to produce a solid gel form of dual drug-loaded MA-PLA₄-PEG₉-PLA₄-MA. The following formulations were prepared 2.5 wt % bupivacaine/2.5 wt % ketorolac—loaded hydrogel, 5 wt % bupivacaine/5 wt % ketorolac—loaded hydrogel, 10 wt % bupivacaine/10 wt % ketorolac—loaded hydrogel

### Example 8. Drug Release from Dual Drug-Loaded MA-PLA₄-PEG₉-PLA₄-MA

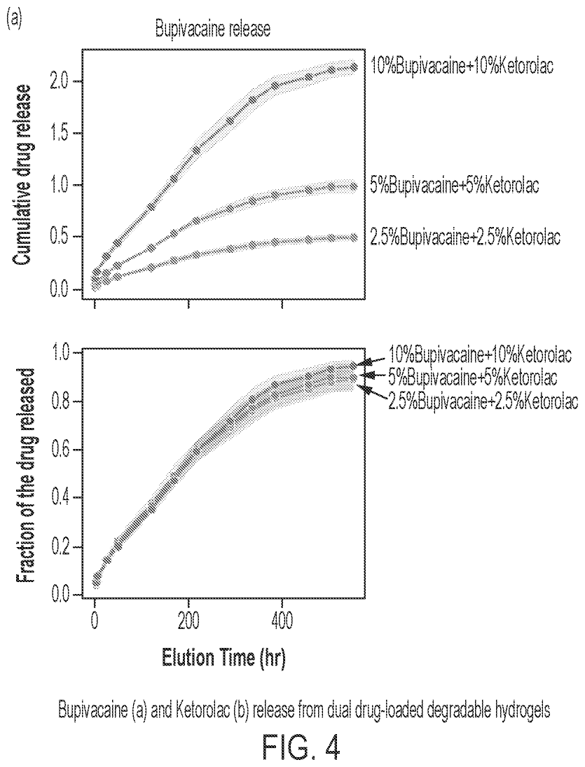

Ketorolac and Bupivacaine-loaded MA-PLA₄-PEG₉-PLA4-MA gels, described in example 7, with dimensions 3 mm×5 mm×20 mm were transferred into 2 ml of PBS. At given time points, PBS was replaced and the concentration of the eluted drugs in the solution was measured using high-performance liquid chromatography. The results of this experiment are in FIG. 4.

### TABLES

TABLE 1
MIC of the tested drugs against MSSA (ATCC 12600).

| Drug | MIC, mg/ml |
|---|---|
| Gentamicin | 0.004 |
| Lidocaine | 15.625 |
| Ketorolac | 3.125 |
| Tolfenamic Acid | 0.25 |
| Bupivacaine | 1.875 |
| Tetracaine | 0.9375 |
| Procaine | 100 |
| Prilocaine | 25 |
| Vancomycin | 0.000625 |
| Curcumin | 0.1875 |
| Resveratrol | 0.125 |

TABLE 2
FIC indices measured against MSSA (ATCC 12600) for
various drug combinations.

| Drug combination | FIC index | Result |
|---|---|---|
| Bupivacaine/Lidocaine | 0.375 | Synergy |
| Bupivacaine/Prilocaine | 1 | Additive effect |
| Bupivacaine/Procaine | 0.75 | Additive effect |
| Bupivacaine/Tetracaine | 0.5 | Synergy |
| Bupivacaine/Vancomycin | 0.7504 | Additive effect |
| Curcumin/Bupivacaine | 1.125 | Additive effect |
| Curcumin/Gentamicin | 0.28125 | Synergy |
| Curcumin/Ketorolac | 0.625 | Synergy |
| Curcumin/Prilocaine | 2.25 | Additive effect |
| Curcumin/Procaine | 2 | Additive effect |
| Curcumin/Tetracaine | 1.5 | Additive effect |
| Curcumin/Tolfenamic Acid | 0.375 | Synergy |
| Curcumin/Vancomycin | 0.3754 | Synergy |
| Gentamicin/Ketorolac | 0.375 | Synergy |
| Gentamicin/Prilocaine | 2 | Additive effect |
| Gentamicin/Procaine | 1 | Additive effect |
| Ketorolac/Lidocaine | 2 | Additive effect |
| Ketorolac/Prilocaine | 1 | Additive effect |
| Ketorolac/Procaine | 0.75 | Additive effect |
| Ketorolac/Tetracaine | 2 | Additive effect |
| Lidocaine/Prilocaine | 0.75 | Additive effect |
| Lidocaine/Procaine | 0.5 | Synergy |
| Prilocaine/Tetracaine | 1 | Additive effect |
| Prilocaine/Vancomycin | 2 | Additive effect |
| Procaine/Prilocaine | 1 | Additive effect |
| Procaine/Tetracaine | 1.5 | Additive effect |
| Procaine/Vancomycin | 1.5008 | Additive effect |
| Resveratrol/ Ketorolac | 2 | Additive effect |
| Resveratrol/Bupivacaine | 0.5625 | Synergy |
| Resveratrol/Gentamicin | 0.28125 | Synergy |
| Resveratrol/Lidocaine | 0.75 | Additive effect |
| Resveratrol/Prilocaine | 0.5 | Synergy |
| Resveratrol/Procaine | 0.5 | Synergy |
| Resveratrol/Tetracaine | 0.25 | Synergy |
| Resveratrol/Tolfenamic Acid | 0.5 | Synergy |
| Resveratrol/Vancomycin | 0.3754 | Synergy |
| Tetracaine/Vancomycin | 1 | Additive effect |
| Tolfenamic Acid/Bupivacaine | 1 | Additive effect |
| Tolfenamic Acid/Gentamicin | 0.375 | Synergy |
| Tolfenamic Acid/Prilocaine | 1 | Additive effect |
| Tolfenamic Acid/Procaine | 0.3125 | Synergy |

TABLE 3
FIC indices measured against MSSA (ATCC 14775)
for various drug combinations

| Drug combination | FIC index | Result |
|---|---|---|
| Bupivacaine/Ketorolac | 2 | Additive effect |
| Bupivacaine/Lidocaine | 0.31 | Synergy |
| Lidocaine/Ketorolac | 2 | Additive effect |
| Bupivacaine/Gentamicin | 0.75 | Additive effect |
| Lidocaine/Gentamicin | 0.75 | Additive effect |
| Ketorolac/Gentamicin | 0.37 | Synergy |

TABLE 4
FIC indices measured against S. epidermidis (ATCC 12228)
for various drug combinations.

| Drug combination | FIC index | Result |
|---|---|---|
| Bupivacaine/Vancomycin | 1.2504 | Additive effect |
| Bupivacaine/Tetracaine | 3 | Additive effect |
| Tolfenamic Acid/Bupivacaine | 0.75 | Additive effect |
| Resveratrol/Lidocaine | 0.375 | Synergy |
| Resveratrol/Bupivacaine | 0.079 | Synergy |
| Resveratrol/Tetracaine | 0.7492 | Additive effect |
| Resveratrol/Vancomycin | 0.75 | Additive effect |
| Resveratrol/Gentamicin | 1 | Additive effect |
| Resveratrol/Tolfenamic Acid | 0.5 | Synergy |

What is claimed is:

1. A method of making an antibacterial polymeric material wherein the method comprises:
a. Providing a first polymeric material,
b. Providing two or more therapeutic agents with synergistic antibacterial activity;
c. Blending the polymeric material with the therapeutic agents, thereby making a therapeutic agent-blended polymeric material,
d. Processing the therapeutic agent-blended polymeric material, thereby making a processed antibacterial, polymeric material.

2. A method of making an antibacterial medical implant wherein the method comprises:
a. Providing a first polymeric material,
b. Providing two or more therapeutic agents with synergistic antibacterial activity;
c. Blending the polymeric material with the therapeutic agents, thereby making a therapeutic agent-blended polymeric material,
d. Processing the therapeutic agent-blended polymeric material, thereby making a processed antibacterial, polymeric material
e. Fashioning a medical implant from the processed antibacterial, polymeric material, thereby making an antibacterial medical implant.

3. A method of making an antibacterial polymeric material wherein the method comprises:
a. Providing a first polymeric material,
b. Providing an antibiotic and a sodium-channel blocker with synergistic antibacterial activity;
c. Blending the polymeric material with at least the antibiotic and the sodium-channel blocker, thereby making a therapeutic agent-blended polymeric material,
d. Processing the therapeutic agent-blended polymeric material, thereby making a processed antibacterial, polymeric material.

4. A method of making an antibacterial polymeric material of claim 3, wherein the provided polymeric material is an ultra-high molecular weight polyethylene.

5. A method of making an antibacterial polymeric material of claim 3, wherein the antibiotic is gentamicin.

6. A method of making an antibacterial polymeric material of claim 3, wherein the sodium-channel blocker is bupivacaine.

7. A method of making an antibacterial polymeric material of claim 3, wherein the processing of the therapeutic agent-blended polymeric material is done by compression molding.

8. A method of making an antibacterial polymeric material of claim 3, wherein the processing of the therapeutic agent-blended polymeric material is photopolymerization.

9. A method of making an antibacterial polymeric material wherein the method comprises:
a. Providing a first polymeric material,
b. Providing an antibiotic and a NSAID with synergistic antibacterial activity;
c. Blending the polymeric material with at least the antibiotic and the NSAID, thereby making a therapeutic agent-blended polymeric material,
d. Processing the therapeutic agent-blended polymeric material, thereby making a processed antibacterial, polymeric material.

10. A method of making an antibacterial polymeric material of claim 9, wherein the provided polymeric material is an ultra-high molecular weight polyethylene.

11. A method of making an antibacterial polymeric material of claim 9, wherein the antibiotic is gentamicin.

12. A method of making an antibacterial polymeric material of claim 9, wherein the NSAID is ketorolac.

13. A method of making an antibacterial polymeric material of claim 9, wherein the NSAID is tolfenamic acid.

14. A method of making an antibacterial polymeric material of claim 9, wherein the processing of the therapeutic agent-blended polymeric material is done by compression molding.

15. A method of making an antibacterial polymeric material of claim 9, wherein the processing of the therapeutic agent-blended polymeric material is photopolymerization.

16. A method of making an antibacterial polymeric material wherein the method comprises:
a. Providing a first polymeric material,
b. Providing an NSAID and a sodium-channel blocker with synergistic antibacterial activity;
c. Blending the polymeric material with at least the sodium-channel blocker and the NSAID, thereby making a therapeutic agent-blended polymeric material,
d. Processing the therapeutic agent-blended polymeric material, thereby making a processed antibacterial, polymeric material.

17. A method of making an antibacterial polymeric material of claim 16, wherein the provided polymeric material is an ultra-high molecular weight polyethylene.

18. A method of making an antibacterial polymeric material of claim 16, wherein the sodium-channel blocker is procaine.

19. A method of making an antibacterial polymeric material of claim 16, wherein the NSAID is tolfenamic acid.

20. A method of making an antibacterial polymeric material of claim 16, wherein the processing of the therapeutic agent-blended polymeric material is done by compression molding.

21. A method of making an antibacterial polymeric material of claim 16, wherein the processing of the therapeutic agent-blended polymeric material is photopolymerization.

22. A method of making an antibacterial polymeric material wherein the method comprises:
a. Providing a first polymeric material,
b. Providing an antioxidant and a sodium-channel blocker with synergistic antibacterial activity;
c. Blending the polymeric material with at least the sodium-channel blocker and antioxidant, thereby making a therapeutic agent-blended polymeric material,
d. Processing the therapeutic agent-blended polymeric material, thereby making a processed antibacterial, polymeric material.

23. A method of making an antibacterial polymeric material of claim 22, wherein the provided polymeric material is an ultra-high molecular weight polyethylene.

24. A method of making an antibacterial polymeric material of claim 22, wherein the antioxidant is resveratrol.

25. A method of making an antibacterial polymeric material of claim 22, wherein the antioxidant is curcumin.

26. A method of making an antibacterial polymeric material of claim 22, wherein the sodium-channel blocker is lidocaine.

27. A method of making an antibacterial polymeric material of claim 22, wherein the sodium-channel blocker is bupivacaine.

28. A method of making an antibacterial polymeric material of claim 22, wherein the sodium-channel blocker is prilocaine.

29. A method of making an antibacterial polymeric material of claim 22, wherein the sodium-channel blocker is procaine.

30. A method of making an antibacterial polymeric material of claim 22, wherein the processing of the therapeutic agent-blended polymeric material is done by compression molding.

31. A method of making an antibacterial polymeric material of claim 22, wherein the processing of the therapeutic agent-blended polymeric material is photopolymerization.

32. A method of making an antibacterial polymeric material wherein the method comprises:
a. Providing a first polymeric material,
b. Providing an NSAID and an antioxidant with synergistic antibacterial activity;
c. Blending the polymeric material with at least the antioxidant and a NSAID, thereby making a therapeutic agent-blended polymeric material,
d. Processing the therapeutic agent-blended polymeric material, thereby making a processed antibacterial, polymeric material.

33. A method of making an antibacterial polymeric material of claim 32, wherein the provided polymeric material is an ultra-high molecular weight polyethylene.

34. A method of making an antibacterial polymeric material of claim 32, wherein the antioxidant is curcumin.

35. A method of making an antibacterial polymeric material of claim 32, wherein the antioxidant is resveratrol.

36. A method of making an antibacterial polymeric material of claim 32, wherein the NSAID is tolfenamic acid.

37. A method of making an antibacterial polymeric material of claim 32, wherein the NSAID is ketorolac.

38. A method of making an antibacterial polymeric material of claim 32, wherein the processing of the therapeutic agent-blended polymeric material is done by compression molding.

39. A method of making an antibacterial polymeric material of claim 32, wherein the processing of the therapeutic agent-blended polymeric material is photopolymerization.

40. A method of making an antibacterial polymeric material wherein the method comprises:
a. Providing a first polymeric material,
b. Providing an antibiotic and an antioxidant with synergistic antibacterial activity;
c. Blending the polymeric material with at least the antioxidant and the antibiotic, thereby making a therapeutic agent-blended polymeric material,
d. Processing the therapeutic agent-blended polymeric material, thereby making a processed antibacterial, polymeric material.

41. A method of making an antibacterial polymeric material of claim 40, wherein the provided polymeric material is an ultra-high molecular weight polyethylene.

42. A method of making an antibacterial polymeric material of claim 40, wherein the antioxidant is curcumin.

43. A method of making an antibacterial polymeric material of claim 40, wherein the antioxidant is resveratrol.

44. A method of making an antibacterial polymeric material of claim 40, wherein the antibiotic is gentamicin.

45. A method of making an antibacterial polymeric material of claim 40, wherein the antibiotic is vancomycin.

46. A method of making an antibacterial polymeric material of claim 40, wherein the processing of the therapeutic agent-blended polymeric material is done by compression molding.

47. A method of making an antibacterial polymeric material of claim 40, wherein the processing of the therapeutic agent-blended polymeric material is photopolymerization.

48. A method of making an antibacterial polymeric material wherein the method comprises:
a. Providing a first polymeric material,
b. Providing two different sodium-channel blockers with synergistic antibacterial activity;
c. Blending the polymeric material with at least two different sodium-channel blockers, thereby making a therapeutic agent-blended polymeric material,
d. Processing the therapeutic agent-blended polymeric material, thereby making a processed antibacterial, polymeric material.

49. A method of making an antibacterial polymeric material of claim 48, wherein the provided polymeric material is an ultra-high molecular weight polyethylene.

50. A method of making an antibacterial polymeric material of claim 48, wherein the sodium-channel blocker is lidocaine, or bupivacaine, or tetracaine, procaine, or prilocaine.

51. A method of making an antibacterial polymeric material of claim 48, wherein the processing of the therapeutic agent-blended polymeric material is done by compression molding.

52. A method of making an antibacterial polymeric material of claim 48, wherein the processing of the therapeutic agent-blended polymeric material is photopolymerization.

53. A method of making an implant comprising:
a. Providing a polymeric material;
b. Blending the polymeric material with two different drugs, including sodium-channel blocker, NSAID, antibiotic, antioxidant;
c. Providing a second polymeric material;
d. Blending the second polymeric material with a cross-linking agent;
e. Layering the dual drug-blended polymeric material and the cross-linking agent-blended second polymeric material;
f. Consolidating the layered polymeric materials; thereby obtaining a cross-linked implant with antibiotic-rich regions.

54. A method of making an implant comprising:
a. Providing a polymeric material;
b. Blending the polymeric material with two different drugs, including sodium-channel blocker, NSAID, antibiotic, antioxidant;
c. Layering the dual drug-blended polymeric material polymeric and the polymeric material without drugs;
d. Consolidating the layered polymeric materials; thereby obtaining an implant with drug-rich regions;
e. Exposing at least parts of the implant to radiation.

55. A method of making a degradable, additive-blended polymeric material comprising:
a. Providing liquid polymerizable macromer composed of a cross-linkable moiety ((Bₘ-Aₙ-Bₘ-R) or (R-Bₘ-Aₙ-Bₘ-R)),
b. Blending the liquid, polymerizable mixture with at least two therapeutic agents (Drug X and Drug Y) with synergistic antibacterial activity
c. Exposing the additive-blended, liquid polymerizable macromer(s) to as external stimulus which can create free radicals for a period of time, thereby forming a degradable, additive-blended gel.

56. A method of making an antibacterial polymeric material of claim 55, wherein the drug X can be sodium-channel blocker, or antibiotic, or anesthetic, or antioxidant, or NSAID. Drug Y can be sodium-channel blocker, or antibiotic, or anesthetic, or antioxidant, or NSAID.

57. A method of making a degradable, additive-blended medical implant comprising:
a. Providing liquid polymerizable macromer composed of a cross-linkable moiety ((Bₘ-Aₙ-Bₘ-R) or (R-Bₘ-Aₙ-Bₘ-R)),
b. Blending the liquid, polymerizable mixture with at least two therapeutic agents (Drug X and Drug Y) with synergistic antibacterial activity
c. Exposing the additive-blended, liquid polymerizable macromer(s) to as external stimulus which can create free radicals for a period of time, thereby forming a medical implant.

58. A method of making an antibacterial polymeric implant of claim 57, wherein the drug X can be sodium-channel blocker, or antibiotic, or anesthetic, or antioxidant, or NSAID. Drug Y can be sodium-channel blocker, or antibiotic, or anesthetic, or antioxidant, or NSAID.
